(19) United States
(12) Patent Application Publication
(10) Pub. No.: US 2010/0247973 A1
(43) Pub. Date: Sep. 30, 2010

(54) REDUCED-ENVIRONMENTAL-IMPACT MAGNETIC SHEET SYSTEMS

(75) Inventors: Joseph B. Stout, Williamstown, WV (US); Jerry King, Williamstown, WV (US)

Correspondence Address: Stoneman Volk Patent Group, P.O. Box 40070, PHOENIX, AZ, 85067-0070, US

(73) Assignee: MAGNUM MAGNETICS CORPORATION, Marietta, OH (US)

(21) Appl. No.: 12/748,123

(22) Filed: Mar. 26, 2010

Related U.S. Application Data
(60) Provisional application No. 61/164,258, filed on Mar. 27, 2009.
(60) Provisional application No. 61/260,337, filed on Nov. 11, 2009.
(60) Provisional application No. 61/259,528, filed on Nov. 9, 2009.

(51) Int. Cl. G11B 5/702; C12P 1/00; H01F 1/26; G11B 5/70

(52) U.S. Cl. 428/844.5; 252/62.54; 252/62.51 R; 435/41; 428/842

(57) ABSTRACT

A reduced-environmental-impact magnetic-sheet systems, processes and methods comprising flexible magnetic sheet material made with reduced-environmental-impact magnetic products from both pre-consumer and post-consumer recycled products.

### CROSS-REFERENCE TO RELATED APPLICATION

The present application is related to and claims priority from prior provisional application Ser. No. 61/164,258, filed Mar. 27, 2009, entitled “REDUCED ENVIRONMENTAL-IMPACT MAGNETIC SYSTEMS”; and, this application is related to and claims priority from prior provisional application Ser. No. 61/260,337, filed Nov. 11, 2009, entitled “REDUCED ENVIRONMENTAL-IMPACT MAGNETIC SYSTEMS”; and, this application is related to and claims priority from prior provisional application Ser. No. 61/259,528, filed Nov. 9, 2009, entitled “MAGNETIC SHEET RECYCLING SYSTEMS”, the contents of all of which are incorporated herein by this reference and are not admitted to be prior art with respect to the present invention by the mention in this cross-reference section.

### BACKGROUND

This invention relates to providing systems and methods relating to reduced-environmental-impact magnetic products. More particularly, this invention relates to providing a system relating to producing reduced-environmental-impact magnetic-sheet products. Additionally, this invention relates to providing a system for recycling pre-consumer waste materials for reuse in magnetic sheets. Recycling lowers the volume of waste sent to landfills and often provides a good source of raw materials that need less processing to render useable in a specific application.

There is an increased awareness in working to preserve our planet's resources and decreasing the amount of added pollution. It would be useful to continue to find ways to utilize recycled material in the manufacture of products and materials. There are many problems in using recycled materials in the manufacturing process, particularly when such recycled products often are comprised of varying percentages of mixed materials.

To be practical, using recycled materials should allow a manufacturing process to lower consumption of “new” materials by replacing some of the overall materials with recycled materials; merely adding more material to the product does not reduce the consumption of new raw materials. Recycling of printable magnetic materials has heretofore been impractical in that contaminates, often in the form of a printable surface sheet, adversely effects the quality of the resulting product if not removed. Flexible magnetic sheets require a careful balance of materials in order to maintain specific flexibility and magnetic qualities while using a minimal amount of materials. Adding too many contaminates into the manufacturing process of magnetic sheets may result in the need to use more new materials to maintain these qualities, ultimately negating the desired effect of using recycled materials in place of new materials. Therefore, a process is needed which can utilize recycled materials in magnetic sheet manufacturing while minimizing the effects of contaminates on the final product.

In addition, the recycling of some waste materials can provide beneficial reductions in energy, pollution, and use of raw materials. The amount of potentially recyclable material available for use has significantly increased in the last 25 years; however, it is not immediately obvious to product manufactures as to which materials can be utilized without producing a net negative benefit in terms of overall environmental impact and product quality. For example, the cost of collecting, transporting, and reusing some waste plastic is higher than utilizing virgin materials. There are also problems in using recycled materials in manufacturing, particularly when such recycled products comprise varying percentages of mixed materials. Incorrectly formulating the compounds used in the production of flexible magnetic sheet can detrimentally influence the mechanical properties of the finished products.

Flexible magnets are used throughout the world in a wide range of applications including printed advertising, surface decoration, and mechanical components. It would be useful to utilize some recycled materials to reduce the environmental impact associated with their production while at the same time maintaining or reducing overall production costs.

### OBJECTS AND FEATURES OF THE INVENTION

A primary object and feature of the present invention is to provide a system overcoming the above-mentioned problems.

It is a further object and feature of the present invention to provide such a system that provides reduced-environmental-impact magnetic products.

It is a further object and feature of the present invention to provide such a system that provides reduced-environmental-impact magnetic-sheet products.

It is a further object and feature of the present invention to provide such a system to manufacture such reduced-environmental-impact magnetic-sheet products.

It is a further object and feature of the present invention to provide such a system to combine environmentally-friendly products with such reduced-environmental-impact magnetic-sheet products.

Another object and feature of the present invention is to provide such a system that increases adhesion between layers of magnetic-sheet products.

It is a further object and feature of the present invention to provide such a system and method for the efficient removal of a non-magnetic low-density printable facing sheet from a higher-density flexible magnetic substrate.

A further primary object and feature of the present invention is to provide such a system that is efficient, inexpensive, and handy. Other objects and features of this invention will become apparent with reference to the following descriptions.

### SUMMARY OF THE INVENTION

In accordance with a preferred embodiment hereof, this invention provides a magnetic sheet system, relating to enhancing at least one adhesive bond when used with at least one writable material, comprising: at least one magnetic-field-source sheet structured and arranged to provide a magnetic field; wherein such at least one magnetic-field-source sheet comprises at least one bondable surface structured and arranged to assist bonding such at least one magnetic-field-source sheet when used with the at least one writeable material; and at least one adhesion enhancer structured and arranged to enhance the at least one adhesive bond of such at least one magnetic-field-source sheet when used with the at least one writeable material; wherein such at least one adhesion enhancer comprises at least one bio-based material.

Moreover, it provides such a system wherein such at least one bio-based material comprises at least one soy-based material. Additionally, it provides such a system wherein such at least one bio-based material comprises at least one soy-based polyester resin. Also, it provides such a system wherein such at least one bio-based material comprises at least one furan resin. In addition, it provides such a system wherein such at least one soy-based polyester comprises about 98%, by weight, polyester resin. And, it provides such a system wherein such at least one bio-based material comprises at least one furan resin.

Further, it provides such a system further comprising at least one pigment-writable material structured and arranged to accept pigmentation forming indicia comprising the at least one writable material. Even further, it provides such a system wherein such at least one bio-based material comprises at least one soy-based polyester resin. Moreover, it provides such a system wherein such at least one soy-based polyester comprises about 98%, by weight, polyester resin. Additionally, it provides such a system wherein such at least one pigment-writable material comprises vinyl. Also, it provides such a system wherein such at least one pigment-writable material comprises bi-axially oriented polypropylene.

In addition, it provides such a system further comprising at least one adhesive structured and arranged to adhere such at least one magnetic-field-source sheet to such at least one pigment-writable material with the at least one adhesive bond.

In accordance with another preferred embodiment hereof, this invention provides a method, relating to enhancing at least one adhesive bond when used with at least one writable material, comprising the steps of: assisting providing at least one mixture of at least one magnetic material structured and arranged to assist providing a magnetic field, and at least one binder material structured and arranged to bind together such at least one magnetic material; assisting providing at least one adhesion enhancer structured and arranged to enhance the at least one adhesive bond; mixing such at least one adhesive enhancer with such at least one mixture of such at least one magnetic material and such at least one binder material, forming at least one adhesion-enhanced magnetic material; wherein such at least one adhesive enhancer comprises at least one bio-based material; and forming at least one magnetic sheet from such at least one adhesion-enhanced magnetic material; wherein such at least one magnetic sheet is magnetizable to provide a magnetic field; and wherein such at least one magnetic sheet comprises at least one bondable surface structured and arranged to assist bonding such at least one magnetic-field-source sheet when used with the at least one writeable material.

And, it provides such a method wherein such at least one bio-based material comprises at least one soy-based material. Further, it provides such a method wherein such at least one bio-based material comprises at least one soy-based polyester resin. Even further, it provides such a method wherein such at least one bio-based material comprises at least one furan resin. Moreover, it provides such a method wherein such at least one soy-based polyester comprises about 98%, by weight, polyester resin. Additionally, it provides such a method wherein such at least one bio-based material comprises at least one furan resin.

Also, it provides such a method further comprising the step of assisting providing at least one pigment-writable material structured and arranged to accept pigmentation forming indicia comprising the at least one writable material. In addition, it provides such a method wherein such at least one bio-based material comprises at least one soy-based polyester resin. And, it provides such a method wherein such at least one soy-based polyester comprises about 98%, by weight, polyester resin. Further, it provides such a method wherein such at least one pigment-writable material comprises vinyl. Even further, it provides such a method wherein such at least one pigment-writable material comprises bi-axially oriented polypropylene.

Even further, it provides such a method further comprising the step of assisting providing at least one adhesive structured and arranged to adhere such at least one magnetic-field-source sheet to such at least one pigment-writable material with the at least one adhesive bond.

In accordance with another preferred embodiment hereof, this invention provides a method, relating to recycling at least one printable magnetic sheet material having at least one low-destiny printable facing attached to at least one higher-density flexible magnetic substrate using at least one adhesive, such method comprising the steps of: pulverizing such at least one printable magnetic sheet material to form at least one pulverized magnetic sheet composition; submerging such at least one pulverized magnetic sheet composition in at least one immersion liquid to assist separation of such at least one low-destiny printable facing from such at least one higher-density flexible magnetic substrate; while such at least one pulverized magnetic sheet composition is submerged within such at least one immersion liquid, agitating such at least one pulverized magnetic sheet composition to assist such separation; collecting from the surface of such at least one immersion liquid, such at least one low-destiny printable facing separated from such at least one higher-density flexible magnetic substrate; and collecting from below the surface of such at least one immersion liquid, such at least one higher-density flexible magnetic substrate comprising reduced quantities of such at least one low-destiny printable facing. Moreover, it provides such a method wherein: such at least one immersion liquid comprises a temperature of between about 80-degrees Fahrenheit and about the boiling point of such at least one immersion liquid; and such agitating of such at least one pulverized magnetic sheet composition comprises a duration of between about 15 minutes and about 60 minutes.

Additionally, it provides such a method wherein such at least one immersion liquid comprises substantially water. Also, it provides such a method wherein such at least one immersion liquid comprises at least one solution of water and at least one enzyme structured and arranged to assist decomposition of the at least one adhesive. In addition, it provides such a method wherein such at least one immersion liquid comprises at least one solution of water and at least one solvent structured and arranged to assist dissolving of the at least one adhesive.

Moreover, it provides such a method further comprising the step of pre-separating such at least one low-destiny printable facing from such at least one higher-density flexible magnetic substrate by passing such at least one pulverized magnetic sheet composition adjacent at least one electrostatically-charged surface structured and arranged to electrostatically attract the at least one low-destiny printable facing. Further, it provides such a method wherein such passage of the at least one pulverized magnetic sheet composition through at least one electrostatically-charged screen occurs prior to the submersing such at least one pulverized magnetic sheet composition in such at least one immersion liquid.

Even further, it provides such a method further comprising the step of removal from such at least one immersion liquid, such at least one adhesive released from such at least one pulverized magnetic sheet composition. Even further, it provides such a method further comprising the step of reusing such at least one higher-density flexible magnetic substrate comprising reduced quantities of such at least one low-destiny printable facing to form at least one new magnetic product.

In accordance with another preferred embodiment hereof, this invention provides a method, relating to recycling at least one printable magnetic sheet material having at least one low-destiny printable facing attached to at least one higher-density flexible magnetic substrate using at least one adhesive, such method comprising the steps of: forming at least one pulverized magnetic sheet composition by pulverizing such at least one printable magnetic sheet material to mechanically detach such at least one low-destiny printable facing from such at least one higher-density flexible magnetic substrate; separating such at least one low-destiny printable facing from such at least one higher-density flexible magnetic substrate by passing such at least one pulverized magnetic sheet composition adjacent at least one electrostatically-charged surface structured and arranged to electrostatically attract the at least one low-destiny printable facing. Moreover, it provides such a method wherein such at least one electrostatically-charged surface comprises at least one separator screen structured and arranged to pass such at least one pulverized magnetic sheet composition. Additionally, it provides such a method further comprising the additional step of separating such at least one low-destiny printable facing from such at least one higher-density flexible magnetic substrate by passing such at least one pulverized magnetic sheet composition adjacent at least one magnetic field structured and arranged to attract such at least one higher-density flexible magnetic substrate and pass such at least one low-destiny printable facing.

In accordance with another preferred embodiment hereof, this invention provides a magnetic sheet system, relating to augmenting magnetic sheets with post-consumer recycling of materials, comprising: at least one magnetic-field-source sheet structured and arranged to provide a magnetic field; wherein such at least one magnetic-field-source sheet comprises at least one binder structured and arranged to bind at least one magnetizable material in such at least one magnetic-field-source sheet; wherein such at least one binder comprises at least one post-consumer recyclable material obtained from the post-consumer recycling. Moreover, it provides such a system wherein such at least one post-consumer recyclable material comprises at least one plastic.

Additionally, it provides such a system wherein such at least one post-consumer recyclable material comprises at least one polyethylene. Also, it provides such a system wherein such at least one post-consumer recyclable material comprises at least one low-density polyethylene. In addition, it provides such a system wherein such at least one binder comprises between about 1% and about 20%, by weight, low-density polyethylene. And, it provides such a system wherein such at least one binder comprises about 1%, by weight, low-density polyethylene.

Further, it provides such a system wherein such at least one binder further comprises: about 40%, by weight, Chlorinated Polyethylene; about 22%, by weight, Ethylene Vinyl Acetate; about 11%, by weight, Polyolefin Elastomer; about 12%, by weight, Polyisobutylene; and about 14%, by weight, Polyolefin Copolymer. Even further, it provides such a system wherein such at least one binder comprises about 7%, by weight, low-density polyethylene. Moreover, it provides such a system wherein such at least one binder further comprises: about 39%, by weight, Chlorinated Polyethylene; about 15%, by weight, Ethylene Vinyl Acetate; about 13%, by weight, Polyolefin Elastomer; about 12%, by weight, Polyisobutylene; and about 14%, by weight, Polyolefin Copolymer. Additionally, it provides such a system wherein such at least one binder comprises about 13%, by weight, low-density polyethylene. Also, it provides such a system wherein such at least one binder further comprises: about 39%, by weight, Chlorinated Polyethylene; about 13%, by weight, Ethylene Vinyl Acetate; about 9%, by weight, Polyolefin Elastomer; about 12%, by weight, Polyisobutylene; and about 14%, by weight, Polyolefin Copolymer.

In addition, it provides such a system further comprising at least one pigment-writable material structured and arranged to accept pigmentation forming indicia comprising the at least one writable material. And, it provides such a system wherein such at least one post-consumer recyclable material comprises at least one low-density polyethylene. Further, it provides such a system wherein such at least one binder comprises between about 1% and about 20%, by weight, low-density polyethylene. Even further, it provides such a system wherein such at least one binder comprises about 1%, by weight, low-density polyethylene. Moreover, it provides such a system wherein such at least one binder comprises about 7%, by weight, low-density polyethylene. Additionally, it provides such a system wherein such at least one binder comprises about 13%, by weight, low-density polyethylene.

Also, it provides such a system wherein such at least one pigment-writable material comprises vinyl. In addition, it provides such a system wherein such at least one pigment-writable material comprises bi-axially oriented polypropylene. And, it provides such a system further comprising at least one adhesive structured and arranged to adhere such at least one magnetic-field-source sheet to such at least one pigment-writable material with the at least one adhesive bond.

In accordance with another preferred embodiment hereof, this invention provides a method, relating to augmenting magnetic sheets with post-consumer recycling of materials, by at least one magnetic-sheet producer, comprising the steps of: assisting providing at least one mixture of at least one magnetic material structured and arranged to assist providing a magnetic field, and at least one binder material structured and arranged to bind together such at least one magnetic material; assisting providing at least one post-consumer recyclable material obtained from the post-consumer recycling; mixing such at least one post-consumer recyclable material with such at least one mixture of such at least one magnetic material and such at least one binder material, forming at least one post-consumer-content magnetic material; forming at least one magnetic sheet from such at least one post-consumer-content magnetic material; and wherein such at least one magnetic sheet is magnetizable to provide a magnetic field. Further, it provides such a method further comprising the steps of: calculating acceptable acquisition costs associated with the acquisition of such at least one post-consumer recyclable material; identifying a plurality of sources of such at least one post-consumer recyclable material; selecting from such plurality of sources, at least one source of such at least one post-consumer recyclable material comprising such acceptable acquisition costs; and arranging for the acquisition of such at least one post-consumer recyclable material from the at least one source selected from such plurality.

Even further, it provides such a method wherein the step of arranging for the acquisition of such at least one post-consumer recyclable material from the at least one source selected from such plurality further comprises the steps of: identifying at least one waste handler providing waste handling within a geographical region encompassing the at least one source and the site of such mixing of such at least one post-consumer recyclable material with such at least one mixture; forming between the at least one magnetic-sheet producer and the at least one waste handler, at least one contract to transport such at least one post-consumer recyclable material between the at least one source and the site of such mixing. Moreover, it provides such a method wherein such at least one contract comprising at least one non-compete agreement binding the at least one waste handler against engaging in competing acquisitions, of such at least one post-consumer recyclable material, on behalf other magnetic-sheet producers.

Additionally, it provides such a method further comprising the steps of: providing pre-qualifying testing of such at least one post-consumer recyclable material received from the at least one source; rejecting off-specification portions of such at least one post-consumer recyclable material. Also, it provides such a method wherein such at least one post-consumer recyclable material comprises at least one plastic. In addition, it provides such a method wherein such at least one post-consumer recyclable material comprises at least one polyethylene. And, it provides such a method wherein such at least one post-consumer recyclable material comprises at least one low-density polyethylene. Further, it provides such a method wherein such at least one binder comprises between about 1% and about 20%, by weight, of such at least one low-density polyethylene.

Even further, it provides such a method wherein such at least one binder comprises about 1%, by weight, low-density polyethylene. Even further, it provides such a method wherein such at least one binder further comprises: about 40%, by weight, Chlorinated Polyethylene; about 22%, by weight, Ethylene Vinyl Acetate; about 11%, by weight, Polyolefin Elastomer; about 12%, by weight, Polyisobutylene; and about 14%, by weight, Polyolefin Copolymer. Even further, it provides such a method wherein such at least one binder comprises about 7%, by weight, low-density polyethylene. Even further, it provides such a method wherein such at least one binder further comprises: about 40%, by weight, Chlorinated Polyethylene; about 22%, by weight, Ethylene Vinyl Acetate; about 11%, by weight, Polyolefin Elastomer; about 12%, by weight, Polyisobutylene; and about 14%, by weight, Polyolefin Copolymer. Even further, it provides such a method wherein such at least one binder comprises about 13%, by weight, low-density polyethylene. Even further, it provides such a method wherein such at least one binder further comprises: about 40%, by weight, Chlorinated Polyethylene; about 22%, by weight, Ethylene Vinyl Acetate; about 11%, by weight, Polyolefin Elastomer; about 12%, by weight, Polyisobutylene; and about 14%, by weight, Polyolefin Copolymer.

Even further, it provides such a method further comprising the steps of: assisting providing at least one pigment-writable material structured and arranged to accept pigmentation forming indicia comprising the at least one writable material; and joining such at least one pigment-writable material with such at least one magnetic sheet. Even further, it provides such a method wherein such at least one post-consumer recyclable material comprises at least one low-density polyethylene. Even further, it provides such a method wherein such at least one binder comprises between about 1% and about 20%, by weight, low-density polyethylene.

Even further, it provides such a method wherein such at least one binder comprises about 1%, by weight, low-density polyethylene. Even further, it provides such a method wherein such at least one binder comprises about 7%, by weight, low-density polyethylene. Even further, it provides such a method wherein such at least one binder comprises about 13%, by weight, low-density polyethylene. Even further, it provides such a method wherein such at least one pigment-writable material comprises vinyl. Even further, it provides such a method wherein such at least one pigment-writable material comprises bi-axially oriented polypropylene.

Even further, it provides such a method further comprising the step of assisting providing at least one adhesive structured and arranged to adhere such at least one magnetic-field-source sheet to such at least one pigment-writable material with the at least one adhesive bond.

In accordance with another preferred embodiment hereof, this invention provides for each and every novel feature, element, combination, step and/or method disclosed or suggested by this patent application.

### BRIEF DESCRIPTION OF THE DRAWINGS

FIG. 1 shows a side view, illustrating a preferred flexible magnetic sheet embodiment, generated by preferred compositions and processes of the present invention.

FIG. 2 shows a sectional view through section 2-2 of FIG. 1 illustrating the preferred construction of the flexible magnetic-field-source sheet.

FIG. 3 shows a diagram view, illustrating at least one recyclable content manufacturing process flow, according to the preferred embodiment of FIG. 1.

FIG. 4 shows a side view, illustrating at least one magnetic sheet, according to a preferred embodiment of the present invention.

FIG. 5 shows a schematic diagram, illustrating methods of recycling waste printable magnetic sheet materials for reuse in new flexible magnetic products, according to a preferred embodiment of the present invention.

FIG. 6 shows a partial sectional view, illustrating the construction of the waste printable magnetic sheet materials, according to the preferred embodiment of FIG. 5.

### DETAILED DESCRIPTION OF THE BEST MODES AND PREFERRED EMBODIMENTS OF THE INVENTION

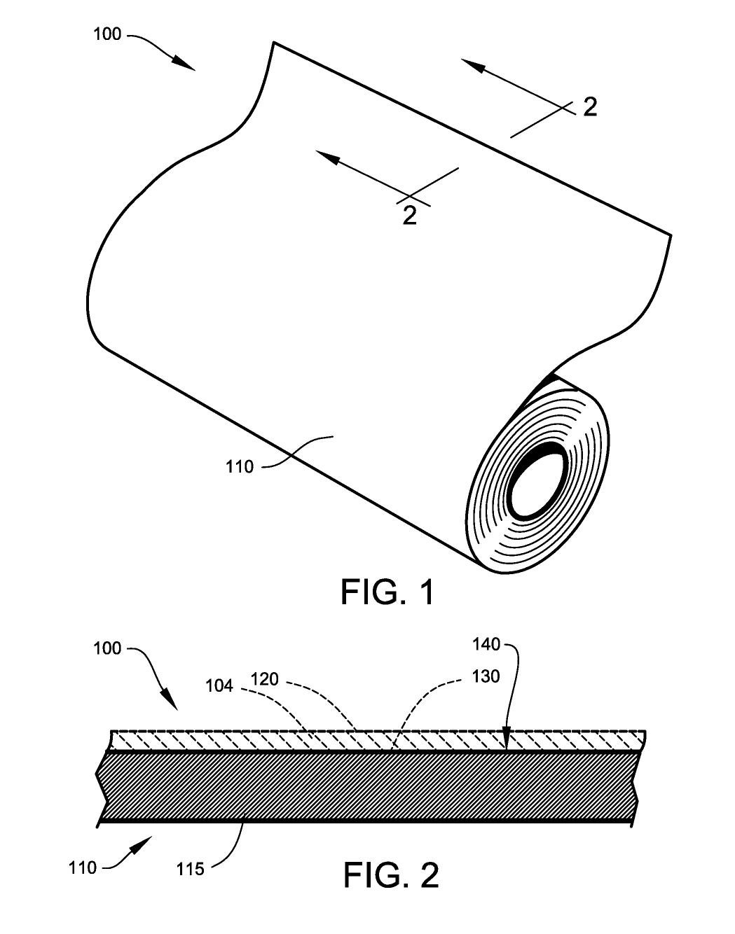

FIG. 1 shows a perspective view, illustrating a preferred flexible magnetic sheet embodiment, generated by preferred compositions and processes of the present invention. FIG. 2 shows a sectional view through section 2-2 of FIG. 1 illustrating the preferred construction of the flexible magnetic-field-source sheet. Preferably, magnetic sheet system 100 comprises at least one flexible magnetic sheet 110, as shown.

Preferably, magnetic sheet system 100 comprises magnetic material manufactured using recycled materials. Such recycled material preferably comprises from about 50% to about 100% pre-consumer recycled content, most preferably from about 70% pre-consumer recycled content to about 80% pre-consumer recycled content. Alternately, such recycled material preferably comprises from about 1% to about 20% post-consumer recycled content. For purposes of this application pre-consumer recycled materials are those recycled materials made with industry scraps or waste from “clean” manufacturing. Post-consumer recycled materials are those materials made from recycled material typically gathered from residents and businesses after use. Those with ordinary skill in the art will now appreciate that upon reading this specification and by their understanding the art of recycling as described herein, methods of re-acquiring and processing recycled materials for re-use will be understood by those knowledgeable in such art.

Further, upon reading this specification, those with ordinary skill in the art will now appreciate that, under appropriate circumstances, considering such issues as design preference, user preferences, marketing preferences, cost, structural requirements, available materials, technological advances, etc., other recycled content arrangements for magnetic sheet materials such as, for example, other recycled magnetic material, higher or lower concentration of pre-consumer or post-consumer recycled waste, etc., may suffice.

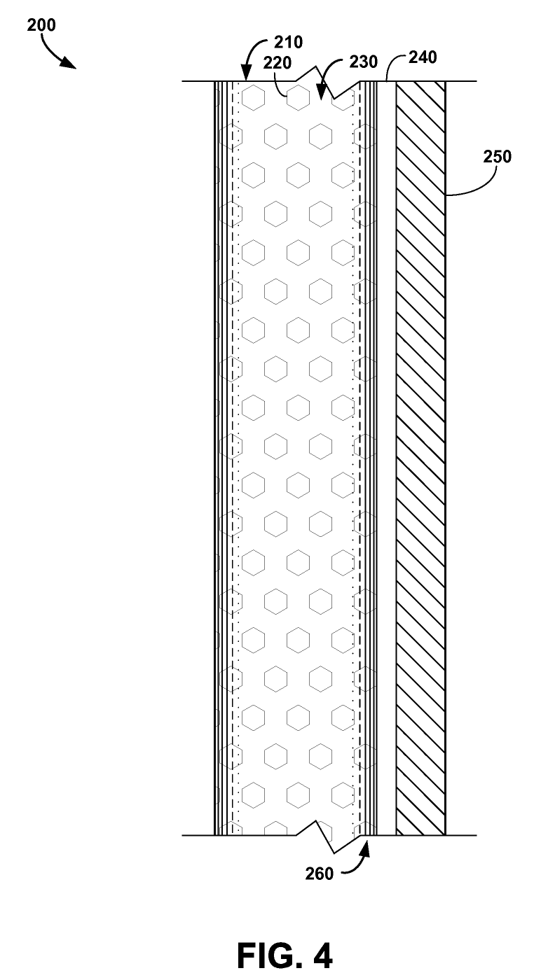

In one embodiment of the present invention (see FIG. 4), magnetic sheet system 100 preferably comprises fully biodegradable materials. Magnet material preferably does not contain petroleum-based derivatives. Magnet materials preferably comprise derivatives from soy, corn or other renewable resources. Bonding agents and adhesives are preferably also made from derivatives from soy, corn or other renewable resources and not petroleum based. Upon reading this specification, those with ordinary skill in the art will now appreciate that, under appropriate circumstances, considering such issues as design preference, user preferences, marketing preferences, cost, structural requirements, available materials, technological advances, etc., other renewable resource materials may suffice.

Magnetic sheet system 100 preferably comprises at least one layer, identified herein as printable face sheet 120 (see the dashed depiction of FIG. 2), of material 104 preferably laminated onto flexible magnetic sheet 110. Material 104 preferably comprises paper, preferably made from 100% post-consumer waste recycled material. Further, such paper materials are preferably made acid-free, chlorine-free and lignin-free. Even further, printable face sheet 120 preferably is capable of comprising indicia, preferably printed indicia, preferably at least inkjet, flexo or offset print applied. Upon reading this specification, those with ordinary skill in the art will now appreciate that, under appropriate circumstances, considering such issues as design preference, user preferences, marketing preferences, cost, structural requirements, available materials, technological advances, etc., other printing arrangements such as, for example, roller, laser, painted, etc., may suffice.

Even further, in flexible magnetic sheet 110 of magnetic sheet system 100, printable face sheet 120 alternately preferably comprises regular paper, alternately preferably polypropylene laminate, alternately preferably vinyl laminate. Such regular paper, such polypropylene and such vinyl laminate each preferably also comprise recycled material. Upon reading the teachings of this specification, those skilled in the art will now appreciate that, under appropriate circumstances, considering such issues as cost, future technologies, etc., other materials, such as, for example, other plastics, cloth, etc., may suffice.

Magnetic sheet system 100 preferably comprises at least one flexible magnet 115, preferably comprising thicknesses from about 12 mil (0.30 mm) to about 30 mil (0.76 mm, millimeter) and preferably laminate layer (printable face sheet 120), preferably about Emil (0.15 mm) in thickness.

Upon reading this specification, those with ordinary skill in the art will now appreciate that, under appropriate circumstances, considering such issues as design preference, user preferences, marketing preferences, cost, structural requirements, available materials, technological advances, etc., other thickness arrangements such as, for example, thinner or thicker, etc., may suffice.

In addition to utilizing recycled materials, it is preferable that magnetic sheet system 100 comprise magnetic materials that meet or exceed child safety standards (for example, such standards as set by an Authority having jurisdiction, such as the U.S. Consumer Product Safety Commission, etc.).

Magnetic sheet system 100 preferably comprises a set of material formulations, enabling the production of flexible sheet-type magnets, preferably using various percentages of recycled material. Such formulations are preferably used to produce the magnetic-field-source sheet of FIG. 1 identified herein as flexible magnetic sheet 110. In one embodiment, flexible magnetic sheet 110 preferably comprises between about 1 percent and about 20 percent post-consumer recycled content by weight. For the purposes of this application, post-consumer recycled materials comprise those materials gathered from one or more sources after the primary use of the material has been completed. Upon reading this specification, those with ordinary skill in the art will now appreciate that, under appropriate circumstances, considering such issues as design preference, user preferences, marketing preferences, cost, structural requirements, available materials, technological advances, etc., other recycled content arrangements for the magnetic sheet materials such as, for example, the use of other recycled magnetic materials, the use of higher or lower concentrations of pre-consumer or post-consumer recycled waste materials, etc., may suffice.

Flexible magnetic sheet materials are typically produced by mixing a powdered ferrite material with a polymeric or plastic binder. It is noted that each of the preferred formulations is preferably adapted to provide a flexible matrix binding to the magnetizable material within flexible magnetic sheet 110 (at least embodying herein wherein such at least one magnetic-field-source sheet comprises at least one binder structured and arranged to bind at least one magnetizable material in such at least one magnetic-field-source sheet; and at least embodying herein at least one magnetic-field-source sheet structured and arranged to provide a magnetic field). Forming of the resultant composition into a planar sheet may preferably be accomplished through the use of a sheet extrusion process and/or calendering.

Careful consideration was given by Applicant to the preferred formulations of the flexible sheet materials containing recycled content. Through experimentation, Applicant discovered a set of preferred formulations, preferably utilizing post-consumer recycled waste materials, which maintained acceptable mechanical properties in the finished product (e.g., flexibility, stability, durability, processability, output rates, etc.). In each of the preferred example formulations below, the binder comprises at least one post-consumer recyclable material obtained from post-consumer recycling. More specifically, such at least one post-consumer recyclable material preferably comprises at least one plastic, preferably at least one polyethylene, preferably at least one low-density polyethylene (LDPE), such total at least one post-consumer recyclable material being preferably in the amount of between about 1% and about 20%, by weight. This arrangement at least herein embodies wherein said at least one magnetic-field-source sheet comprises at least one binder structured and arranged to bind at least one magnetizable material in said at least one magnetic-field-source sheet; and this arrangement at least herein embodies wherein said at least one binder comprises at least one post-consumer recyclable material obtained from the post-consumer recycling.

Outlined below are three preferred flexible magnet binder formulations using recycled low density polyethylene as the post-consumer recycled content.

### Example 1

### High Load LDPE Binder Formula 300A

| Material | Grade | Weight (lbs) |
|---|---|---|
| CPE | Tyrin (CM3031 MP) | 34.00 |
| EVA | MI = 2, VA = 18%, Escorene Ultra | 11.00 |
| | LD 728 | |
| Engage | 8200 | 8.00 |
| Vistanex | P118-EF | 10.90 |
| Vistamax | V6102 | 12.10 |
| LDPE | Post Consumer | 11.00 |
| Total | | 87.00 |

### Example 2

### Low Load LDPE Binder Formula 300B

| Material | Grade | Weight (lbs) |
|---|---|---|
| CPE | Tyrin (CM3031 MP) | 34.00 |
| EVA | MI = 2, VA = 18%, Escorene Ultra | 13.00 |
| | LD 728 | |
| Engage | 8200 | 11.00 |
| Vistanex | P118-EF | 10.90 |
| Vistamax | V6102 | 12.10 |
| LDPE | Post Consumer | 6.00 |
| Total | | 87.00 |

### Example 3

### Premix 1050 with LDPE

### Binder Formula 300C

| Material | Grade | Weight (lbs) |
|---|---|---|
| CPE | Tyrin (CM3031 MP) | 35.00 |
| EVA | MI = 2, VA = 18%, Escorene Ultra | 19.09 |
| | LD 728 | |
| Engage | 8200 | 9.90 |
| Vistanex | P118-EF | 10.90 |
| Vistamaxx | V6102 | 12.10 |
| Recycled LDPE | Post Consumer | 1.00 |
| Total | | 87.99 |

In the above examples:

CPE is Chlorinated Polyethylene

EVA is Ethylene Vinyl Acetate

Engage® is a Polyolefin Elastomer

Vistanex® is Polyisobutylene

Vistamaxx™ is a Polyolefin Copolymer

It is again noted that each of the above preferred example formulations (Binder Formulas 300A through 300C) is preferably adapted to form a flexible matrix binding the magnetizable material within flexible magnetic sheet 110 (at least embodying herein wherein such at least one magnetic-field-source sheet comprises at least one binder structured and arranged to bind at least one magnetizable material in such at least one magnetic-field-source sheet).

In specific reference to binder formula 300A (Example 1), the preferred formulation comprises 13%, by weight, low-density polyethylene (LDPE); about 39%, by weight, Chlorinated Polyethylene; about 13%, by weight, Ethylene Vinyl Acetate; about 9%, by weight, Polyolefin Elastomer; about 12%, by weight, Polyisobutylene; and about 14%, by weight, Polyolefin Copolymer.

LDPE materials for binder formula 300A preferably comprise low-density polyethylene derived from recycled materials. The LDPE materials are preferably shredded (reground) prior to use. Material properties of flexible magnetic sheet 110 produced from binder formula 300A are shown in Table A of this specification.

TABLE A
Material Properties

| Sample ID | After Test | Formula 300A | Formula 300B |
|---|---|---|---|
| Date | | | |
| Production Date | | | |
| Powder HOLM Lot(s) and | # Lot 540 Bag 61 | # Lot 540 Bag 61 | # Lot 540 Bag 61 |
| Amount | | | |
| TDK and Amount | # Lot 3132 Sk 13 | # Lot 3132 Sk 13 | # Lot 3132 Sk 13 |
| Formula/Binder Amount | 1053 | | |
| Mixer | 1 | 1 | 1 |
| Calendar | 1 | 2 | 2 |
| Millage | 0.012 | 0.011 | 0.011 |
| Br | 1598 | 1633 | 1680 |
| Hc | 1147 | 1140 | 1146 |
| Hci | 1960 | 1893 | 1869 |
| Bh max | 0.53 | 0.54 | 0.57 |
| Density | 3.48 | 3.49 | 3.51 |
| Shore D | 55 | 55 | 55 |
| | Initial Tensile | Initial Tensile | Initial Tensile |
| Tensile Strength | n/a | n/a | n/a |
| Elongation at Break | n/a | n/a | n/a |
| Yield Strength | n/a | n/a | n/a |
| Elongation at Yield | n/a | n/a | n/a |
| | 24 Hour Tensile | 24 Hour Tensile | 24 Hour Tensile |
| Tensile Strength | 606 psi | 686 psi | 513 psi |
| Elongation at Break | 15.5% | 26.5% | 9.0% |
| Yield Strength | 825 psi | 809 psi | 812 psi |
| Elongation at Yield | 6.0% | 9.8% | 5.6% |
| Zero AG | 0 | 0 | 0 |
| 035″ AG | 0 | 0 | 0 |
| Block | n/a | n/a | n/a |
| Severe Fold CD | ok | ok | ok |
| Severe Fold MD | ok | ok | ok |
| Fold on ¼″ Rod CD | ok | ok | ok |
| Fold on ¼″ Rod MD | ok | ok | ok |
| Magnetizer | 0 | 0 | 0 |

In specific reference to binder formula 300B (Example 2), the preferred formulation comprises about 7%, by weight, LDPE; about 39%, by weight, Chlorinated Polyethylene; about 15%, by weight, Ethylene Vinyl Acetate; about 13%, by weight, Polyolefin Elastomer; about 12%, by weight, Polyisobutylene; and about 14%, by weight, Polyolefin Copolymer.

LDPE materials for binder formula 300B preferably comprise low-density polyethylene derived from recycled materials. The LDPE materials are preferably shredded (reground) prior to use. Material properties of flexible magnetic sheet 110 produced from binder formula 300B are shown in Table A of this specification.

TABLE B
Material Properties

| Sample ID | Formula 300C | Before | |
|---|---|---|---|
| Powder HSR Lot(s) and | Lot 1146 Bag 3 300# | Lot 1146 Bag 3 | Random Sample Made |
| Amount | | 300# | @ 2:30 |
| TDK and Amount | 55# | 55# | |
| Formula/Binder Amount | MF 1050 w/LDPE | MF 1050 | MF 1050 |
| Mixer | 2 | 2 | 2 |
| Calendar | 4 | 4 | 4 |
| Millage | 0.029 | 0.031 | 0.0295 |
| Br | 1622 | 1578 | 1680 |
| Hc | 1095 | 1051 | 1210 |
| Hci | 1796 | 1733 | 2092 |
| Bh max | 0.51 | 0.47 | 0.57 |
| Density | 3.57 | 3.59 | 3.55 |
| Shore D | 62 | 63 | 61 |

Initial Tensile

| | | | |
|---|---|---|---|
| Tensile Strength | 736 psi | 702 psi | 839 psi |
| Elongation at Break | 31.0% | 17.9% | 43.0% |
| Yield Strength | 815 psi | 870 psi | 791 psi |
| Elongation at Yield | 7.8% | 7.8% | 8.9% |

24 Hour Tensile

| | | | |
|---|---|---|---|
| Tensile Strength | 785 psi | 799 psi | 789 psi |
| Elongation at Break | 27.8% | 13.7% | 37.3% |
| Yield Strength | 833 psi | 903 psi | 835 psi |
| Elongation at Yield | 7.2% | 7.8% | 8.1% |
| Zero AG | 0 | 0 | 0 |
| 035″ AG | 0 | 0 | 0 |
| Block | .7370 lbs/2 in | 0.7593 lbs/2 in | n/a |
| Severe Fold CD | Good | Cracks | Good |
| Severe Fold MD | slight pinholes | Cracks | Good |
| Fold on ¼″ Rod CD | Good | Good | Good |
| Fold on ¼″ Rod MD | Good | cracks | Good |
| Magnetizer | 0 | 0 | 0 |

| | |
|---|---|
| Comment | Before Test “Elong @ Break” is unexpectedly low. |

In specific reference to binder formula 300C (Example 3), the preferred formulation comprises about 1%, by weight, LDPE combined with a premixed formula of about 40%, by weight, Chlorinated Polyethylene; about 22%, by weight, Ethylene Vinyl Acetate; about 11%, by weight, Polyolefin Elastomer; about 12%, by weight, Polyisobutylene; and about 14%, by weight, Polyolefin Copolymer.

LDPE materials for binder formula 300C preferably comprise low-density polyethylene derived from recycled materials. The LDPE materials are preferably shredded (reground) prior to use. Material properties of flexible magnetic sheet 110 produced from binder formula 300C are shown in Table B of the specification.

To produce flexible magnetic sheet 110 of FIG. 1, one or more of the above-described preferred binder formulations are preferably mixed with at least one magnetic material. Such magnetic materials preferably comprise at least one substance capable of producing a magnetic field, preferably strontium ferrite, alternately preferably barium ferrite. Upon reading the teachings of this specification, those skilled in the art will now appreciate that, under appropriate circumstances, considering such issues as cost, future technologies, etc., other magnetic materials, such as, for example, neodymium, iron, other rare earth magnetic materials, other ferrous materials, etc., may suffice.

The mixture of binder and magnetic material is preferably formed into a flexible sheet or roll, as shown. Forming of mixture into a planar sheet is preferably accomplished through one or more production processes, such as, for example, by calendering and/or the use of a thermal sheet extrusion process. The finished flexible magnetic 115 is preferably capable of comprising a thickness range of between about 12 mil (0.30 mm) and about 30 mil (0.76 mm, millimeter).

Preferred embodiments of magnetic sheet system 100 comprise at least one pigment-writable material, identified herein as printable face sheet 120 (see the dashed depiction of FIG. 2), preferably bonded to flexible magnetic 115 using at least one adhesive 130. Each of the above-noted formulations produces a suitable bonding surface 140 for receiving such adhesives and pigment-writable materials.

Printable face sheet 120 is preferably structured and arranged to accept printing, writing, or similar pigmentation forming indicia on the material. Preferred materials compositions for printable face sheet 120 comprise paper, alternately preferably vinyl, alternately preferably bi-axially oriented polypropylene.

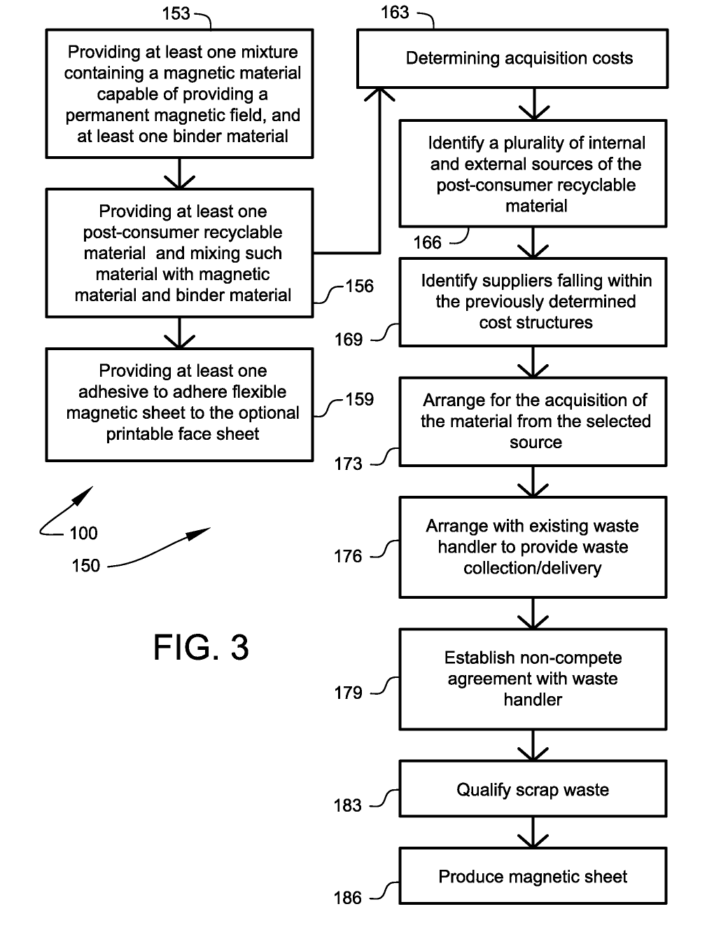

FIG. 3 shows a diagram view, illustrating at least one recyclable content manufacturing process flow, according to the preferred embodiment of FIG. 1. In accordance with magnetic sheet system 100 there is provided a preferred method 150 relating to augmenting magnetic sheets with post-consumer recycling of materials, by at least one magnetic-sheet producer, comprising the steps of: preferably assisting in providing at least one mixture containing a magnetic material capable of providing a permanent magnetic field, and at least one binder material (preferably adapted to bind together the magnetic material), in step 153; preferably assisting in providing at least one post-consumer recyclable material preferably obtained from the post-consumer recycling, in step 156; preferably mixing such post-consumer recyclable material or materials with the mixture of magnetic material and binder material, in step 156, to generate a post-consumer-content magnetic material. This material composition is preferably used to form flexible magnetic 115. In the above-described method 150, flexible magnetic 115 is preferably magnetizable to provide a durable magnetic field. Method 150 further comprises the preferred step of assisting providing adhesive 130 structured and arranged to adhere flexible magnetic 115 to the optional printable face sheet 120 (e.g., such at least one pigment-writable material), preferably forming flexible magnetic sheet 110 (see step 186).

Method 150 further preferably comprises a series of preferred steps associated with the acquisition of the post-consumer recyclable materials by the magnetic-sheet producer. These preferred steps preferably include the calculating of acceptable costs associated with the acquisition and processing of the post-consumer recyclable materials (step 163).

The preferred step of determining acquisition costs preferably include analysis of availability of “on-site” materials, analysis of bulk purchase prices from external sources, handling and shipping costs between an external source and the processing site, testing costs, pre-processing, etc. Once a framework of acceptable acquisition costs has been identified, the magnetic-sheet producer preferably identifies a plurality of internal and external sources of the post-consumer recyclable material, in step 166. Internal sources preferably include waste LDPE generated by onsite processes of the magnetic-sheet producer. Preferred external sources of waste LDPE include nearby industrial operations, alternately preferably, local and regional recycled-waste processors, etc. Upon reading this specification, those with ordinary skill in the art will now appreciate that, under appropriate circumstances, considering such issues as cost, availability of waste materials, material quality requirements, etc., other acquisition arrangements such as, for example, sourcing from brokers of off-spec pre-consumer materials, etc., may suffice.

Based on acquisition costs, the plurality of potential sources will be narrowed to those sources and suppliers falling within the previously determined cost structures, in step 169. For example, selected sources may reside in a geographical region offering a relatively short shipping distance from the production site of the magnetic-sheet producer.

Next the magnetic-sheet producer preferably arranges for the acquisition of the post-consumer recyclable material from the selected source or sources, in step 173. Preferred step 173 of method 150 may preferably involve on-site sorting and collection of waste materials produced by the magnetic-sheet producer (e.g., providing sorting bins for the collection of shipping scrap). Alternately preferably, the magnetic-sheet producer may preferably establish a means for collecting and delivering waste materials from a source located remotely. Thus, magnetic-sheet producer may preferably provide sorting bins to other off-site waste-generating operations, preferably followed by regular collection and delivery of the collected waste to the producer's processing site.

Alternately preferably, the magnetic-sheet producer may arrange with one or more existing waste handlers to provide waste handling (collection and delivery) within a geographical region encompassing the LDPE waste source and the producer's processing site (embodying herein the site of such mixing of such at least one post-consumer recyclable material with such at least one mixture). A preferred waste handler may comprise a private waste management company already operating waste transport trucks within the region. In a preferred step 179 of method 150 a contract is established between the magnetic-sheet producer and the waste handler to transport the consumer recyclable material between the source(s) of the scrap and the producer's processing site. Such a contract preferably comprises at least one non-compete agreement prohibiting the waste handler from engaging in competing acquisitions, of the post-consumer recyclable material, on behalf other magnetic-sheet producers of the region.

On receipt of the post-consumer recyclable material, the magnetic-sheet producer qualifies the scrap material through testing, in step 183. This preferred quality control step of method 150 is intended to identify and reject off-specification portions of the post-consumer recyclable material. Preferably, other common polymers used in the post-consumer recyclable material, such as, for example, ethylene vinyl acetate (EVA) or polyvinyl chloride (PVC) are preferably sorted and removed for disposal or recycling. LDPE materials are preferably retained for reuse. Upon reading this specification, those with ordinary skill in the art will now appreciate that, under appropriate circumstances, considering such issues as cost, advances in polymer technology, etc., other material arrangements such as, for example, utilizing a wider range of plastic materials, etc., may suffice.

FIG. 4 shows a side view, illustrating at least one magnetic sheet 200, according to a preferred embodiment of the present invention. Magnetic sheet 200 of magnetic sheet system 100 preferably comprises fully biodegradable materials. Magnet material preferably does not contain petroleum-based derivatives. Magnet materials preferably comprise derivatives from soy, corn or other renewable resources. Bonding agents and adhesives are preferably also made from derivatives from soy, corn or other renewable resources and not petroleum based. Upon reading this specification, those with ordinary skill in the art will now appreciate that, under appropriate circumstances, considering such issues as design preference, user preferences, marketing preferences, cost, structural requirements, available materials, technological advances, etc., other renewable resource materials may suffice.

During experimental development, Applicant identified a problem associated with maintaining the adhesion of polymeric-type writable materials to the magnetic-sheet substrate, which increased with the use of the above-described environmentally-friendly materials. When such magnetic-sheet product was exposed to outdoor conditions (e.g., simulating use of the product as exterior signage, etc.), a trend toward premature de-lamination of the writable material was observed; such a tendency was found to increase with the use of such environmentally-friendly materials. Therefore, enhancing interfacial adhesion was discovered to be essential in the production of durable exterior-grade products.

To overcome this problem, Applicant tested alternate formulations using magnetic sheet 110 as the basis. Through such experimentation, Applicant discovered an alternate preferred formulation that effectively overcomes the de-lamination issue identified above. This alternate preferred product, identified herein as magnetic sheet 200, preferably comprises the composition of magnetic sheet 110; however, magnetic sheet 200 further comprises at least one adhesion enhancer 260.

Magnetic sheet 200 preferably comprises at least one magnetic-source sheet 210 joined to writable material 250. Magnetic-source sheet 210 preferably comprises at least one magnetic material 220, preferably suspended in at least one binder material 230. Magnetic material 220 preferably comprises at least one material which may produce a magnetic field, preferably strontium ferrite, alternately preferably barium ferrite. Upon reading the teachings of this specification, those skilled in the art will now appreciate that, under appropriate circumstances, considering such issues as cost, future technologies, etc., other magnetic materials, such as, for example, neodymium, iron, other rare earth magnetic materials, other ferrous materials, etc., may suffice.

Writeable material 250 preferably is adhered to magnetic-source sheet 210 (at least embodying herein at least one magnetic-field-source sheet structured and arranged to provide a magnetic field), preferably with at least one adhesive 240. Upon reading the teachings of this specification, those skilled in the art will now appreciate that, under appropriate circumstances, considering such issues as cost, available materials, etc., other bonding agents, such as, for example, material fusing agents, heat, paired materials, etc., may suffice.

Magnetic writable material 220, binder material 230, and adhesive 240 preferably comprise environmentally friendly constituents, preferably made from renewable resources. Upon reading the teachings of this specification, those skilled in the art will now appreciate that, under appropriate circumstances, considering such issues as cost, future materials, etc., other constituent materials, such as, for example, recycled materials, biodegradable materials, recyclable materials, plastics, cloth, etc., may suffice.

Writable material 250 preferably comprises vinyl, alternately preferably bi-axially oriented polypropylene. Upon reading the teachings of this specification, those skilled in the art will now appreciate that, under appropriate circumstances, considering such issues as cost, future materials, etc., other writable materials, such as, for example, paper, other bio-based plastics, cloth, etc., may suffice.

Writable material 250 preferably provides a surface on magnetic sheet 200 which may preferably be written upon, alternately preferably printed upon. Writable material 250 preferably accepts pigmentation from at least one writing implement, alternately preferably from at least one printer. Upon reading the teachings of this specification, those skilled in the art will now appreciate that, under appropriate circumstances, considering such issues as cost, future materials, application, etc., other methods of pigmentation, such as, for example, silk screening, painting, etc., may suffice.

Adhesion enhancer 260 preferably increases adhesion of adhesive 240 to magnetic-source sheet 210, preferably preventing de-lamination of magnetic-source sheet 210 and writable material 250, preferably in an outdoor environment. Adhesion enhancer 260 preferably is added to binder material 230 during manufacture of magnetic-source sheet 210. At manufacture, adhesion enhancer 260 preferably is mixed, preferably uniformly, with binder material 230 and magnetic material 220. By the time writable material 250 (at least embodying herein at least one pigment-writable material structured and arranged to accept pigmentation forming indicia comprising the at least one writable material) is adhered to magnetic-source sheet 210, a significant portion of adhesion enhancer 260 (at least embodying herein at least one adhesion enhancer structured and arranged to enhance the at least one adhesive bond of said at least one magnetic-field-source sheet when used with the at least one writeable material) will have migrated to the surface (at least herein embodying wherein said at least one magnetic-field-source sheet comprises at least one bondable surface structured and arranged to assist bonding said at least one magnetic-field-source sheet when used with the at least one writeable material) of magnetic-source sheet 210. Upon reading the teachings of this specification, those skilled in the art will now appreciate that, under appropriate circumstances, considering such issues as cost, future technologies, etc., other adhesion enhancer implementations, such as, for example, surface coating, high velocity implantation, high pressure application, etc., may suffice.

Adhesion enhancer 260 preferably comprises at least one adhesive, preferably at least one resin, preferably at least one bio-based resin, preferably at least one soy-based resin, preferably at least one furan soy-based resin, preferably at least one soy-based polyester resin, preferably at least one soy-based polyester resin comprising about 98% polyester, preferably BioRez® 13062 available from Advanced Image Resources, LLC of Alpharetta, Ga. This arrangement at least herein embodies wherein said at least one adhesion enhancer comprises at least one bio-based material. Upon reading the teachings of this specification, those skilled in the art will now appreciate that, under appropriate circumstances, considering such issues as cost, future technologies, etc., other resins, such as, for example, corn-based resins, other vegetable-based resins, other furan resins, other polyester resins, etc., may suffice.

Adhesion enhancer preferably comprises from about 2 percent to about 5 percent, by volume, of magnetic-source sheet 210. Upon reading the teachings of this specification, those skilled in the art will now appreciate that, under appropriate circumstances, considering such issues as cost, available materials, etc., other amounts of adhesion enhancer, such as, for example, less than about 2 percent, about 5 percent to about 10 percent, greater than 10 percent, etc., may suffice.

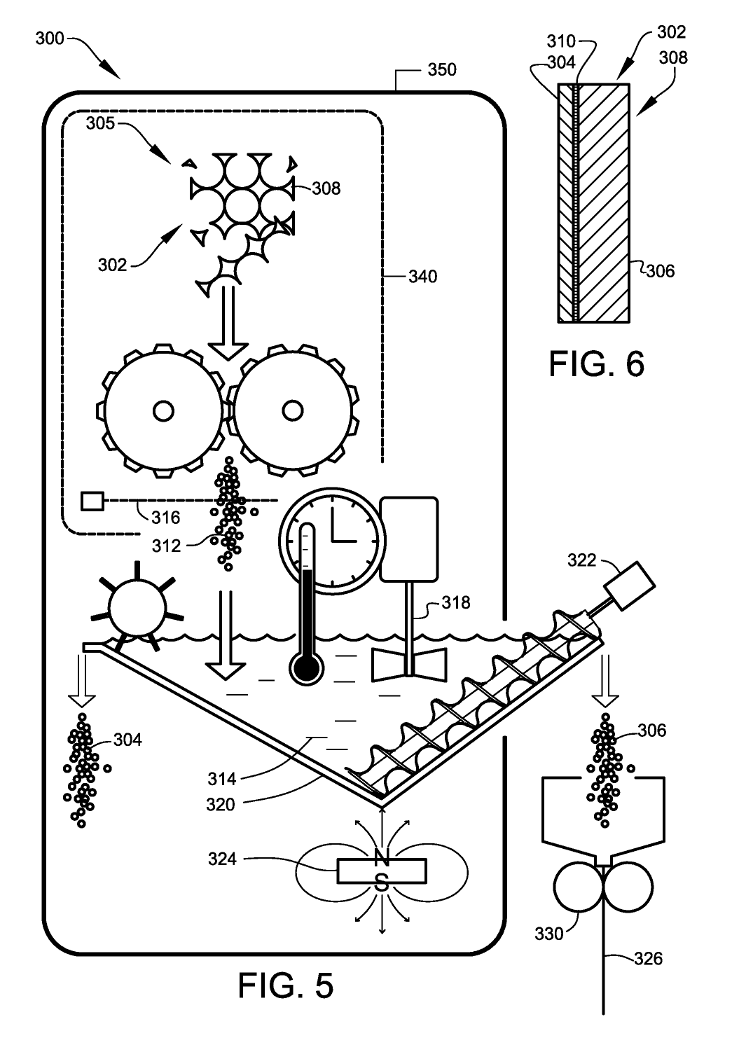

FIG. 5 shows a schematic diagram, illustrating methods of recycling waste writeable magnetic sheet material 302 for reuse in new flexible magnetic products, according to a preferred embodiment of the present invention. FIG. 6 shows a partial sectional view, illustrating the construction of waste writeable magnetic sheet material 302, according to the preferred embodiment of FIG. 5.

The preferred processes of the present invention provide a means for reclaiming waste writeable magnetic sheet material 302 for use in new flexible magnetic products. These materials are typically produced by mixing a powdered ferrite material with a polymeric or plastic binder. Such flexible permanent magnetic materials are often supplied in the form of sheets or rolls. Forming of the materials into a planar sheet may preferably be accomplished through the use of a sheet extrusion process or by calendering. A popular application of such sheet materials is the production of printed magnets having on their outer surfaces promotional or decorative markings, including advertisements, business cards, calendars, greeting cards, entertaining indicia, and the like.

Most magnetic sheet materials are inherently dark in color and it is therefore usual to bond a light-colored printable face sheet onto these magnetic sheets. The resulting printable magnetic sheet material 308 (substantially similar to flexible magnetic sheet 110 and magnetic sheet 200) can be made into printed products of any desired shape by cutting and/or stamping. This processing often produces significant amounts of unused waste trimmings 305. Unused waste trimmings 305 are preferably efficiently reclaimed by the preferred processes of the present invention.

As best illustrated in FIG. 6, printable magnetic sheet material 308 is preferably formed by firmly bonding the non-magnetic low-destiny printable facing 304 to the higher-density flexible magnetic substrate 306. The printable facing and magnetic substrate are typically joined using an interfacial adhesive 310, as shown. Printable facing 304 may comprise a range of preferred materials, such as, for example, paper, vinyl, or polypropylene. For such waste recycling to be practical, most of the low-destiny printable facing 304 must be removed from the higher-density flexible magnetic substrate 306 prior to reuse. This requires the bonding adhesive 310 to be weakened sufficiently to allow delaminating of the facing and substrate.

With respect to the preferred processes schematically illustrated in FIG. 5, magnetic sheet recycling system 300 comprises a first method 350 relating to recycling of printable magnetic sheet material 308 through the removal of the low-destiny printable facing 304 from the higher-density flexible magnetic substrate. In the initial preferred step of method 350, waste printable magnetic sheet material 308 is preferably pulverized to produce a pulverized magnetic sheet composition 312, as shown. The pulverization of the material functions to mechanically loosen and separate a portion of printable facing 304 from flexible magnetic substrate 306. This arrangement at least embodies herein pulverizing such at least one printable magnetic sheet material to form at least one pulverized magnetic sheet composition.

In addition, pulverization reduces printable magnetic sheet material 308 to particles having a size of not more than about ¼ inch. This preferred size improves the effectiveness of the subsequent preferred step wherein the pulverized magnetic sheet composition 312 is placed in an immersion liquid 314 used to break down adhesive 310, as shown. This arrangement at least embodies herein submerging such at least one pulverized magnetic sheet composition in at least one immersion liquid to assist separation of such at least one low-destiny printable facing from such at least one higher-density flexible magnetic substrate.

In an optional preferred step of method 350, pulverized magnetic sheet composition 312 is passed adjacent an electrostatically-charged surface 316 preferably structured and arranged to attract and separate the low-destiny printable facing 304 from the higher-density flexible magnetic substrate. In a preferred embodiment of the system, electrostatically-charged surface 316 comprises a metallic screen preferably adapted to pass the granular flexible magnetic substrate 306 while retaining the relatively lightweight printable facing 304 by electrostatic attraction. This arrangement at least embodies herein pre-separating such at least one low-destiny printable facing from such at least one higher-density flexible magnetic substrate by passing such at least one pulverized magnetic sheet composition adjacent at least one electrostatically-charged surface structured and arranged to electrostatically attract the at least one low-destiny printable facing. Upon reading this specification, those with ordinary skill in the art will now appreciate that, under appropriate circumstances, considering such issues as cost, material composition, end use, etc., other pre-separation arrangements such as, for example, magnetic-assisted pre-separation, low-temperature fracturing, blower-assisted pre-separation, etc., may suffice.

In the immersion step of method 350, the pulverized magnetic sheet composition 312 is soaked in immersion liquid 314 to induce the degradation of adhesive 310. In most cases, it is preferred that immersion liquid 314 comprises water or, alternately preferably, an aqueous solution preferably containing an enzyme (at least herein embodying wherein such at least one immersion liquid comprises at least one solution of water and at least one enzyme structured and arranged to assist decomposition of the at least one adhesive) or, alternately preferably, one or more chemical solvent additives (at least herein embodying wherein such at least one immersion liquid comprises at least one solution of water and at least one solvent structured and arranged to assist dissolving of the at least one adhesive) assisting the dispersion of adhesive 310. The preferred reduced particle size produced during pulverization allows immersion liquid 314 to more easily migrate across the entire interfacial adhesive region during soaking

Immersion liquid 314 is preferably maintained at a constant temperature ranging between about 80-degrees Fahrenheit and less than about the boiling point of immersion liquid 314. In a preferred embodiment of the system, immersion liquid 314 is preferably maintained at a constant temperature of about 155-degrees Fahrenheit. Upon reading this specification, those with ordinary skill in the art will now appreciate that, under appropriate circumstances, considering such issues as thermal stability of materials, adhesive composition/bond strength, aqueous composition, etc., other temperature arrangements such as, for example, utilizing boiling temperatures, etc., may suffice.

Pulverized magnetic sheet composition 312 is maintained in immersion liquid 314 for a preferred duration of between about 15 minutes and 120 minutes. During experimental testing, Applicant noted an association between liquid temperature and time required to produce acceptable levels of delamination; specifically, higher liquid temperatures correlated to reduced immersion times. A preferred time/temperature relationship comprises the use of a 155-degree immersion liquid and an immersion time of about 15 minutes.

At least one agitator 318 is used to continuously agitate pulverized magnetic sheet composition 312 during the emersion step. In a preferred embodiment of the present system, agitator 318 comprises mechanically-driven stir paddle. Alternately preferably, agitator 318 comprises a vibratory device structured and arranged to vibrate the holding tank 320 containing immersion liquid 314 and pulverized magnetic sheet composition 312. This arrangement at least embodies herein while such at least one pulverized magnetic sheet composition is submerged within such at least one immersion liquid, agitating such at least one pulverized magnetic sheet composition to assist such separation. Upon reading this specification, those with ordinary skill in the art will now appreciate that, under appropriate circumstances, considering such issues as material composition, advances in technology, etc., other agitation arrangements such as, for example, the use of gas spargers, liquid circulation pumps, tumble chambers, etc., may suffice.

As delamination occurs, the low-density printable facing 304 floats to the surface of immersion liquid 314 and is preferably removed from tank 320 by surface skimming, as shown. The collected printable facing 304 is preferably and saved for disposal or possible recycling. This arrangement at least embodies herein collecting, from the surface of such at least one immersion liquid, such at least one low-destiny printable facing separated from such at least one higher-density flexible magnetic substrate.

At the end of the immersion step, agitation is preferably stopped allowing the denser flexible magnetic substrate 306 to settle to the bottom of tank 320. Settling and collection is preferably facilitated by the preferred introduction of at least one magnetic field source 324 adjacent the bottom of tank 320. Magnetic field source 324 preferably assists in drawing the smaller magnetically attractive particles out of suspension toward the collection point at the bottom of tank 320, as shown.

Once sufficient settling has occurred, one or more extractors 322 are preferably used to recover flexible magnetic substrate 306 from the bottom of tank 320, as shown. The recovered flexible magnetic substrate 306 is now significantly free of printable facing 304 and is ready for recycling and subsequent reprocessing. This arrangement at least embodies herein collecting, from below the surface of such at least one immersion liquid, such at least one higher-density flexible magnetic substrate; and this arrangement at least embodies herein wherein such at least one higher-density flexible magnetic substrate comprises a recyclable state having reduced material contamination from presence of such at least one low-destiny printable facing.

In a subsequent preferred step of method 350, flexible magnetic substrate 306 is used to form at least one new magnetic product 326, as shown. To produce such products, the recovered flexible magnetic substrate 306 may be mixed with virgin materials including compatible polymer binders, dispersing agents, wetting agents, etc. The resulting thermoplastic composition may be melted and reformed using calendaring processes. This arrangement at least embodies herein reusing such at least one higher-density flexible magnetic substrate comprising reduced quantities of such at least one low-destiny printable facing to form at least one new magnetic product.

In an alternate preferred step of method 350, adhesive 310 released from pulverized magnetic sheet composition 312 is recovered from immersion liquid 314. Such recovery enhances water conservation and reduces environmental pollution. Recovery may be undertaken using known methods including filter separation, chemical-induced flocculation, thermal processing, etc. This arrangement at least embodies herein removal from such at least one immersion liquid, such at least one adhesive released from such at least one pulverized magnetic sheet composition.

In alternate preferred method (similar to method 340) of magnetic sheet recycling system 300, the separation of printable facing 304 from flexible magnetic substrate 306 occurs entirely by electrostatic separation, as indicated in by the inset boundary of FIG. 1. Such alternate preferred method is useful when the mechanical properties of printable magnetic sheet material 308 are such that significant delamination of printable facing 304 from flexible magnetic substrate 306 occurs during the above-described pulverization step.

Although applicant has described applicant's preferred embodiments of this invention, it will be understood that the broadest scope of this invention includes modifications such as diverse shapes, sizes, and materials. Such scope is limited only by the below claims as read in connection with the above specification. Further, many other advantages of applicant's invention will be apparent to those skilled in the art from the above descriptions and the below claims.

What is claimed is:

1) A method, relating to recycling at least one printable magnetic sheet material having at least one low-destiny printable facing attached to at least one higher-density flexible magnetic substrate using at least one adhesive, said method comprising the steps of:
a) pulverizing such at least one printable magnetic sheet material to form at least one pulverized magnetic sheet composition;
b) submerging such at least one pulverized magnetic sheet composition in at least one immersion liquid to assist separation of such at least one low-destiny printable facing from such at least one higher-density flexible magnetic substrate;
c) while such at least one pulverized magnetic sheet composition is submerged within such at least one immersion liquid, agitating such at least one pulverized magnetic sheet composition to assist such separation;
d) collecting, from the surface of such at least one immersion liquid, such at least one low-destiny printable facing separated from such at least one higher-density flexible magnetic substrate; and
e) collecting, from below the surface of such at least one immersion liquid, such at least one higher-density flexible magnetic substrate;
f) wherein such at least one higher-density flexible magnetic substrate comprises a recyclable state having reduced material contamination from presence of such at least one low-destiny printable facing.

2) method according to claim 1 wherein such at least one immersion liquid comprises a temperature of between about 80-degrees Fahrenheit and about the boiling point of such at least one immersion liquid; and

3) The method according to claim 2 wherein such agitating of such at least one pulverized magnetic sheet composition comprises a duration of between about 15 minutes and about 60 minutes.

4) method according to claim 1 wherein such at least one immersion liquid comprises substantially water.

5) method according to claim 1 wherein such at least one immersion liquid comprises at least one solution of water and at least one enzyme structured and arranged to assist decomposition of the at least one adhesive.

6) method according to claim 1 wherein such at least one immersion liquid comprises at least one solution of water and at least one solvent structured and arranged to assist dissolving of the at least one adhesive.

7) method according to claim 1 further comprising the step of pre-separating such at least one low-destiny printable facing from such at least one higher-density flexible magnetic substrate by passing such at least one pulverized magnetic sheet composition adjacent at least one electrostatically-charged surface structured and arranged to electrostatically attract the at least one low-destiny printable facing.

8) method according to claim 7 wherein such passage of the at least one pulverized magnetic sheet composition adjacent such at least one electrostatically-charged surface occurs prior to the step of submersing such at least one pulverized magnetic sheet composition in such at least one immersion liquid.

9) method according to claim 1 further comprising the additional step of separating such at least one low-destiny printable facing from such at least one higher-density flexible magnetic substrate by passing such at least one pulverized magnetic sheet composition adjacent at least one magnetic field structured and arranged to attract such at least one higher-density flexible magnetic substrate and pass such at least one low-destiny printable facing.

10) method according to claim 1 further comprising the step of removal from such at least one immersion liquid, such at least one adhesive released from such at least one pulverized magnetic sheet composition.

11) method according to claim 1 further comprising the step of reusing such at least one higher-density flexible magnetic substrate comprising reduced quantities of such at least one low-destiny printable facing to form at least one new magnetic product.

12) A method, relating to recycling at least one printable magnetic sheet material having at least one low-destiny printable facing attached to at least one higher-density flexible magnetic substrate using at least one adhesive, said method comprising the steps of:
a) forming at least one pulverized magnetic sheet composition by pulverizing such at least one printable magnetic sheet material to mechanically detach such at least one low-destiny printable facing from such at least one higher-density flexible magnetic substrate;
b) separating such at least one low-destiny printable facing from such at least one higher-density flexible magnetic substrate by passing such at least one pulverized magnetic sheet composition adjacent at least one electrostatically-charged surface structured and arranged to electrostatically attract the at least one low-destiny printable facing.

13) method according to claim 12 wherein such at least one electrostatically-charged surface comprises at least one separator screen structured and arranged to pass such at least one pulverized magnetic sheet composition.

14) method according to claim 12 further comprising the additional step of separating such at least one low-destiny printable facing from such at least one higher-density flexible magnetic substrate by passing such at least one pulverized magnetic sheet composition adjacent at least one magnetic field structured and arranged to attract such at least one higher-density flexible magnetic substrate and pass such at least one low-destiny printable facing.

15) A magnetic sheet system, relating to augmenting magnetic sheets with post-consumer recycling of materials, comprising:
a) at least one magnetic-field-source sheet structured and arranged to provide a magnetic field;
b) wherein said at least one magnetic-field-source sheet comprises at least one binder structured and arranged to bind at least one magnetizable material in said at least one magnetic-field-source sheet;
c) wherein said at least one binder comprises at least one post-consumer recyclable material obtained from the post-consumer recycling.

16) system according to claim 15 wherein said at least one post-consumer recyclable material comprises at least one plastic.

17) system according to claim 16 wherein said at least one post-consumer recyclable material comprises at least one polyethylene.

18) system according to claim 17 wherein said at least one post-consumer recyclable material comprises at least one low-density polyethylene.

19) system according to claim 18 wherein said at least one binder comprises from about 1% to about 20%, by weight, low-density polyethylene.

20) system according to claim 19 wherein said at least one binder comprises about 1%, by weight, low-density polyethylene.

21) system according to claim 20 wherein said at least one binder further comprises:
a) about 40%, by weight, Chlorinated Polyethylene;
b) about 22%, by weight, Ethylene Vinyl Acetate;
c) about 11%, by weight, Polyolefin Elastomer;
d) about 12%, by weight, Polyisobutylene; and
e) about 14%, by weight, Polyolefin Copolymer.

22) system according to claim 19 wherein said at least one binder comprises about 7%, by weight, low-density polyethylene.

23) system according to claim 22 wherein said at least one binder further comprises:
a) about 39%, by weight, Chlorinated Polyethylene;
b) about 15%, by weight, Ethylene Vinyl Acetate;
c) about 13%, by weight, Polyolefin Elastomer;
d) about 12%, by weight, Polyisobutylene; and
e) about 14%, by weight, Polyolefin Copolymer.

24) system according to claim 19 wherein said at least one binder comprises about 13%, by weight, low-density polyethylene.

25) system according to claim 24 wherein said at least one binder further comprises:
a) about 39%, by weight, Chlorinated Polyethylene;
b) about 13%, by weight, Ethylene Vinyl Acetate;
c) about 9%, by weight, Polyolefin Elastomer;
d) about 12%, by weight, Polyisobutylene; and
e) about 14%, by weight, Polyolefin Copolymer.

26) system according to claim 15 further comprising at least one pigment-writable material structured and arranged to accept pigmentation to form indicia.

27) system according to claim 26 wherein said at least one post-consumer recyclable material comprises at least one low-density polyethylene.

28) system according to claim 27 wherein said at least one binder comprises between about 1% and about 20%, by weight, low-density polyethylene.

29) A magnetic sheet system, relating to enhancing at least one adhesive bond when used with at least one writable material, comprising:
a) at least one magnetic-field-source sheet structured and arranged to provide a magnetic field;
b) wherein said at least one magnetic-field-source sheet comprises at least one bondable surface structured and arranged to assist bonding said at least one magnetic-field-source sheet when used with the at least one writeable material; and
c) at least one adhesion enhancer structured and arranged to enhance the at least one adhesive bond of said at least one magnetic-field-source sheet when used with the at least one writeable material;
d) wherein said at least one adhesion enhancer comprises at least one bio-based material.

30) system according to claim 29 wherein said at least one bio-based material comprises at least one soy-based material.

31) system according to claim 30 wherein said at least one bio-based material comprises at least one soy-based polyester resin.

32) system according to claim 31 wherein said at least one bio-based material comprises at least one furan resin.

33) system according to claim 31 wherein said at least one soy-based polyester comprises about 98%, by weight, polyester resin.

34) system according to claim 29 wherein said at least one bio-based material comprises at least one furan resin.

35) system according to claim 29 further comprising at least one pigment-writable material structured and arranged to accept pigmentation forming indicia.

36) system according to claim 35 wherein said at least one bio-based material comprises at least one soy-based polyester resin.

37) system according to claim 36 wherein said at least one soy-based polyester comprises about 98%, by weight, polyester resin.

38) system according to claim 35 wherein said at least one pigment-writable material comprises vinyl.

39) system according to claim 35 wherein said at least one pigment-writable material comprises bi-axially oriented polypropylene.
